(19) United States
(12) Patent Application Publication
(10) Pub. No.: US 2023/0340578 A1
(43) Pub. Date: Oct. 26, 2023

(54) SPATIAL ANALYSIS OF MULTIPLE TARGETS IN TISSUE SAMPLES

(71) Applicant: ROCHE SEQUENCING SOLUTIONS, INC., Pleasanton, CA (US)

(72) Inventors: Jan BERKA, Pleasanton, CA (US); Dieter HEINDL, Penzberg (DE); Sedide OZTURK, Pleasanton, CA (US); Nikolaus-Peter STENGELE, Penzberg (DE)

(21) Appl. No.: 18/012,607

(22) PCT Filed: Jul. 8, 2021
(86) PCT No.: PCT/EP2021/068977

§ 371 Date: Dec. 22, 2022

Related U.S. Application Data
(60) Provisional application No. 63/049,218, filed on Jul. 8, 2020.

(51) Int. Cl. C12Q 1/6841; C12Q 1/6804

(52) U.S. Cl. CPC C12Q 1/6841; C12Q 1/6804

(57) ABSTRACT

The invention provides methods and compositions for analysis of single cell in tissue sample allowing for simultaneous detection and localization of multiple targets in the cells.

### FIELD OF THE INVENTION

The invention relates to the field of biological analysis of single cells and tissues. More specifically, the invention relates to the detecting multiple targets in individual cells of a three-dimensional tissue sample.

### BACKGROUND OF THE INVENTION

Single cell analysis is finding more applications in research and diagnostics. Of special interest is the study of tissues at the cellular level, see e.g., WO2018091676 Method for spatial tagging and analysing nucleic acids in a biological specimen. One new technique is able to detect multiple targets in millions of individual cells. This technique termed “Quantum Barcoding” or “QBC” (U.S. Pat. No. 10,144,950) is easy to use and economical: millions of cells can be analyzed with no special equipment as the output is read by nucleic acid sequencing. At the heart of the QBC method is assembly of combinatorial barcodes unique to each cell via the process of split-pool. Because this process requires pooling, mixing and splitting cell-containing volumes, the process cannot be applied to a fixed structure such as tissue or organ.

There is a need for a method of analyzing individual cells that could be applied to a two-dimensional or a three-dimensional structure such as a tissue slide or an organ.

### SUMMARY OF THE INVENTION

The invention relates to spatial detection of targets in individual cells of a two-dimensional or three-dimensional tissue sample. The invention involves transfer of spatially discrete target-binding events from a tissue sample onto a layer of particles, where each particle carries a location code, and using a unique barcoding method to associate each target-binding event with a location code within the tissue sample.

In some embodiments, the invention is a method of simultaneously detecting the presence and location of multiple targets in a tissue sample, the method comprising: contacting a tissue sample with one or more unique binding agents, wherein the agents include a target-identifying nucleic acid conjugated to a capture moiety; forming on the tissue sample a layer of particles conjugated to a capture molecule capable of selectively binding the capture moiety; contacting the layer of particles with a plurality of location-identifying nucleic acids conjugated to the capture moiety; capturing the target-identifying nucleic acids and the location-identifying nucleic acids on the particles via the capture moiety and separating the particles from the tissue sample into a liquid sample; assembling unique particle-specific codes on each particle-bound target-identifying nucleic acid and each location-identifying nucleic acid by adding to the nucleic acids multiple subcode oligonucleotides in an ordered manner during successive rounds of split-pool synthesis (wherein each round comprises: splitting the liquid sample into reaction volumes, each volume comprising a species of subcode oligonucleotide; annealing the subcode oligonucleotide adjacently to the subcode oligonucleotide from a previous round via an annealing region; covalently linking the adjacently annealed subcode oligonucleotides to each other; and pooling the reaction volumes into a liquid sample) detecting the sequence of the target-identifying nucleic acids and the location-identifying nucleic acids and associated unique particle-specific codes; for each target, correlating the target-identifying nucleic acids and the location-identifying nucleic acids having the same unique particle-specific code thereby detecting the presence and location of multiple targets in the tissue sample. In some embodiments, the unique binding agent is an antibody conjugated to the target-identifying nucleic acid. In some embodiments, the unique binding agent is a nucleic acid probe comprising a target-identifying barcode. In some embodiments, the target-identifying nucleic acid comprises: a first oligonucleotide including a target-identifying barcode; and a second oligonucleotide hybridized to the first oligonucleotide and including the capture moiety and further including the annealing region for attaching subcodes. In some embodiments, the location-identifying nucleic acid comprises: a third oligonucleotide capable of attachment to the surface of the cells and including a location-identifying barcode; and a fourth oligonucleotide hybridized to the third oligonucleotide and including the capture moiety and further including the annealing region for attaching subcodes. In some embodiments, the sample-identifying oligonucleotide is conjugated to a moiety capable of attaching to the surface of the cells in the tissue sample. In some embodiments, the moiety is biotin and the method further comprises coating the surface of the cells with streptavidin prior to contacting the tissue sample with the location-identifying nucleic acid. In some embodiments, the moiety is a fatty acid residue or a cholesterol moiety capable of forming a hydrophobic interaction with the membrane of the cells. In some embodiments, the moiety is a maleimide moiety capable of reacting with amino groups present in cell membrane proteins of the cells. In some embodiments, the moiety is a phosphine moiety capable of reacting with carbohydrate residues associated with cell membrane proteins of the cells. In some embodiments, the capture moiety is biotin and the particle comprises a streptavidin-coated polymer. In some embodiments, the particle has magnetic or paramagnetic properties. In some embodiments, separating the particles from the tissue sample comprises denaturing the hybrids of the first and second oligonucleotides and the hybrids of the third and fourth oligonucleotides. In some embodiments, the particles are separated from solution containing the tissue sample. In some embodiments, the particles are spheres fewer than 10 micrometers in diameter, e.g., fewer than 5 micrometers in diameter.

In some embodiments, the method further comprises washing the tissue sample to remove unbound unique binding agents. In some embodiments, the wash comprises a protease and a detergent or a chaotropic agent.

In some embodiments, the layer of particles is a monolayer. In some embodiments, contacting the layer of particles with location-identifying nucleic acids comprises placing an addressable array of location-identifying nucleic acids atop the layer or particles under conditions suitable for capturing the capture moiety of the nucleic acids with capture molecule on the particles. In some embodiments, the location of the multiple targets is determined in the addressable array. In some embodiments, the density of the array corresponds to the size of the cells so that each particle captures fewer than 5 cells, e.g., no more than 1 cell.

In some embodiments, separating the particles from the tissue sample in step d. comprises treatment with formamide or formamide alternatives selected from sulfolane, ethylene carbonate, pyrrolidone, DMSO or a primary amide.

In some embodiments, the unique binding agent further comprises a sample-identifying nucleic acid and multiple liquid samples are pooled prior to detecting particle-specific barcodes.

In some embodiments, the invention is a method of detecting the presence and location of multiple targets in a tissue sample, the method comprising: contacting a tissue sample with one or more unique binding agents, wherein the agents include a target-identifying nucleic acid conjugated to a capture moiety; a plurality of location-identifying nucleic acids conjugated to the capture moiety; forming on the tissue sample a layer of particles conjugated to a capture molecule capable of selectively binding the capture moiety; capturing the target-identifying nucleic acids and the location-identifying nucleic acids on the particles via the capture moiety and separating the particles from the tissue sample into a liquid sample; assembling unique particle-specific codes on each particle-bound target-identifying nucleic acid and each location-identifying nucleic acid by adding to the nucleic acids multiple subcode oligonucleotides in an ordered manner during successive rounds of split-pool synthesis (wherein each round comprises: splitting the liquid sample into reaction volumes, each volume comprising a species of subcode oligonucleotide; annealing the subcode oligonucleotide adjacently to the subcode oligonucleotide from a previous round via an annealing region; covalently linking the adjacently annealed subcode oligonucleotides to each other; and pooling the reaction volumes into a liquid sample) detecting the sequence of the target-identifying nucleic acids and the location-identifying nucleic acids and associated unique particle-specific codes; for each target, correlating the target-identifying nucleic acids and the location-identifying nucleic acids having the same unique particle-specific code thereby detecting the presence and location of multiple targets in the tissue sample.

In some embodiments, the unique binding agent is an antibody conjugated to the target-identifying nucleic acid. In some embodiments, the unique binding agent is a nucleic acid probe comprising a target-identifying barcode. In some embodiments, the target-identifying nucleic acid comprises: a first oligonucleotide including a target-identifying barcode; and a second oligonucleotide hybridized to the first oligonucleotide and including the capture moiety and further including the annealing region for attaching subcodes. In some embodiments, the location-identifying nucleic acid comprises: a third oligonucleotide capable of attachment to the surface of the cells and including a location-identifying barcode; and a fourth oligonucleotide hybridized to the third oligonucleotide and including the capture moiety and further including the annealing region for attaching subcodes. In some embodiments, the sample-identifying oligonucleotide is conjugated to a moiety capable of attaching to the surface of the cells in the tissue sample. In some embodiments, the moiety is biotin and the method further comprises coating the surface of the cells with streptavidin prior to contacting the tissue sample with the location-identifying nucleic acid. In some embodiments, the moiety is a fatty acid residue or a cholesterol moiety capable of forming a hydrophobic interaction with the membrane of the cells. In some embodiments, the moiety is a maleimide moiety capable of reacting with amino groups present in cell membrane proteins of the cells. In some embodiments, the moiety is a phosphine moiety capable of reacting with carbohydrate residues associated with cell membrane proteins of the cells. In some embodiments, the capture moiety is biotin and the particle comprises a streptavidin-coated polymer.

In some embodiments, the particle has magnetic or paramagnetic properties. In some embodiments, separating the particles from the tissue sample in step d. comprises denaturing the hybrids of the first and second oligonucleotides and the hybrids of the third and fourth oligonucleotides. In some embodiments, the particles are separated from solution containing the tissue sample. In some embodiments, particles are spheres fewer than 10 micrometers in diameter, e.g., fewer than 5 micrometers in diameter.

In some embodiments, the method further comprises washing the tissue sample to remove unbound unique binding agents. In some embodiments, the wash comprises treatment with a protease in the presence of a detergent or a chaotropic agent.

In some embodiments, the layer of particles is a monolayer. In some embodiments, contacting the layer of particles with location-identifying nucleic acids comprises placing an addressable array of location-identifying nucleic acids atop the layer or particles under conditions suitable for capturing the capture moiety of the nucleic acids with capture molecule on the particles. In some embodiments, the location of the multiple targets is determined in the addressable array. In some embodiments, the density of the array corresponds to the size of the cells so that each particle captures fewer than 5 cells, e.g., no more than 1 cell.

In some embodiments, separating the particles from the tissue sample in step d. comprises treatment with formamide or formamide alternatives selected from sulfolane, ethylene carbonate, pyrrolidone, DMSO or a primary amide.

In some embodiments, the unique binding agent further comprises a sample-identifying nucleic acid and multiple liquid samples are pooled prior to detecting particle-specific barcodes.

In some embodiments, the invention is a kit for simultaneously detecting the presence and location of multiple targets in a tissue sample, the kit comprising: one or more unique binding agents, wherein the agents include a target-identifying nucleic acid conjugated to a capture moiety; solid state particles conjugated to a capture molecule capable of selectively binding the capture moiety; a plurality of location-identifying nucleic acids conjugated to the capture moiety; a plurality of subcode oligonucleotides comprising annealing region and a code and reagents for connecting the subcode to each other. In some embodiments, the unique binding agent is an antibody conjugated to the target-identifying nucleic acid. In some embodiments, the unique binding agent is a nucleic acid probe comprising a target-identifying barcode. In some embodiments, the target-identifying nucleic acid comprises: a first oligonucleotide including a target-identifying barcode; and a second oligonucleotide hybridized to the first oligonucleotide and including the capture moiety and further including the annealing region for attaching subcodes. In some embodiments, the location-identifying nucleic acid comprises: a third oligonucleotide capable of attachment to the surface of the cells and including a location-identifying barcode; and a fourth oligonucleotide hybridized to the third oligonucleotide and including the capture moiety and further including the annealing region for attaching subcodes.

In some embodiments, the kit further comprises a sample-identifying oligonucleotide conjugated to a moiety capable of attaching to the surface of the cells in the tissue sample. In some embodiments, the moiety is biotin. In some embodiments, the moiety is a fatty acid residue or a cholesterol moiety capable of forming a hydrophobic interaction with the membrane of the cells. In some embodiments, the moiety is a maleimide moiety capable of reacting with amino groups present in cell membrane proteins of the cells. In some embodiments, the moiety is a phosphine moiety capable of reacting with carbohydrate residues associated with cell membrane proteins of the cells. In some embodiments, the capture moiety is biotin and the particle comprises a streptavidin-coated polymer.

In some embodiments, the particle in the kit has magnetic or paramagnetic properties. In some embodiments, the particles are spheres fewer than 10 micrometers in diameter, e.g., fewer than 5 micrometers in diameter.

In some embodiments, the kit further comprises a protease, a detergent and a chaotropic agent.

In some embodiments of the kit, the location-identifying oligonucleotides are supplied in an addressable array. In some embodiments, the density of the array corresponds to the size of the cells to be analyzed with the kit so that each particle captures fewer than 5 cells, e.g., no more than 1 cell.

In some embodiments, the kit further comprises a solution of formamide or formamide alternatives selected from sulfolane, ethylene carbonate, pyrrolidone, DMSO or a primary amide.

In some embodiments of the kit, the unique binding agent further comprises a sample-identifying nucleic acid and multiple liquid samples are pooled prior to detecting particle-specific barcodes. In some embodiments, the kit further comprises a double-hairpin oligonucleotide for removing excess subcodes from the reaction mixture, the double hairpin nucleic acid comprising a single nucleic acid strand having: a first hairpin at the 5′-end; a second hairpin at the 3′-end; and a single-stranded region between the 5′-end and the 3′-end, wherein the single-stranded region comprises a sequence capable of hybridizing to the subcode oligonucleotide.

In some embodiments of the kit, the capture moiety is a capture sequence on the second and fourth oligonucleotides and the kit comprises particles conjugate to a capture molecule comprising a capture oligonucleotide complementary to the capture sequence.

In some embodiments, the invention is a use of the kit described in the preceding paragraphs to detect location and presence of one or more targets in a tissue sample.

In some embodiments, the method and kit of the invention further comprise a step of removing excess subcode oligonucleotides from a reaction mixture and reagents therefor, the method comprising: after attaching the subcode oligonucleotides to the subcode oligonucleotide of the previous round, contacting the reaction mixture with a double hairpin nucleic acid comprising a single nucleic acid strand having: a first hairpin at the 5′-end; a second hairpin at the 3′-end; and a single-stranded region between the 5′-end and the 3′-end, wherein the single-stranded region comprises a sequence capable of hybridizing to the subcode oligonucleotide; annealing the excess subcode oligonucleotide to the double hairpin nucleic acid; ligating the excess subcode oligonucleotide to the ends of the double hairpin nucleic acid thereby removing the excess subcode oligonucleotide from the reaction mixture. In some embodiments, the capture moiety is a capture sequence at least partially complementary to a capture molecule comprising a capture oligonucleotide.

In some embodiments of the kit, one or both of the capture moiety and the capture molecule include one or more modified nucleotides that alter the melting temperature (Tm) of the duplex DNA selected from 5-methyl cytosine, 2,6-diaminopurine, Super T (5-hydroxybutynl-2-deoxyuridine), Super G (8-aza-7-deazaguanosine, locked nucleic acid (LNA) nucleotides, ribonucleotides and 2′-O-methyl ribonucleotides.

In some embodiments, the invention is a system for detecting the presence and location of multiple targets in a tissue sample, the system comprising a computer with a programmable processor, a memory storage and a graphic display, wherein the programmable processor comprises computer code enabling associating each location-identifying nucleic acid with a location on the array, and further associating each target-identifying nucleic acid with the location-identifying nucleic acid by virtue of sharing a unique particle-associated barcode, thus associating the target with the location in the array, superimposing of the addressable array onto the tissue sample, and generating an image comprising the tissue sample, the array and the location of each target in the array.

### BRIEF DESCRIPTION OF THE DRAWINGS

FIG. 1 is a diagram of one embodiment of the workflow of the invention.

FIG. 2 is an illustration of a tissue sample with a bound antibody and bound location identifiers identifying two distinct locations.

FIG. 3 is an illustration of a combinatorial barcode assembly via rounds of split-pool synthesis.

FIG. 4 is an illustration of a particle with a particle-associated nucleic acid encoding a target code and a location code as well as a unique particle-associated code.

FIG. 5 is a diagram of another embodiment of the workflow of the invention.

### DETAILED DESCRIPTION OF THE INVENTION

The term “nucleic acid” refers to a nucleotide polymer, and unless otherwise limited, includes known analogs of natural nucleotides that can function in a similar manner (e.g., hybridize) to naturally occurring nucleotides.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Polynucleotide sequences, when provided, are listed in the 5′ to 3′ direction, unless stated otherwise.

The term “probe” refers to an oligonucleotide capable of binding to a target nucleic acid generally through complementary base pairing, although perfect complementarity is not required thus forming a duplex structure. The probe binds or hybridizes to a “probe binding site.” The probe can be labeled with a detectable label to permit detection of the probe, particularly once the probe has hybridized to its complementary target. Alternatively, however, the probe may be unlabeled, but may be detectable by specific binding with a ligand that is labeled, either directly or indirectly.

The term “epitope” and “target molecule” are used interchangeably herein to refer to the molecule of interest (parts of it or the whole molecule) being detected and/or quantified by the methods described herein.

The term “barcode” refers to a sequence of nucleotides that tags an entity. A barcode is read by sequencing. A barcode can tag entities that cannot themselves be sequenced by nucleic acid sequencing, e.g., proteins or cells. For example, a unique molecular ID (UID) is a barcode that uniquely tags a nucleic acid molecule. A sample ID (SID) is a barcode that tags all the molecules in a sample to be distinguished from molecules originating from other samples. A target ID or target identifying barcode is a barcode that identifies a target molecule, e.g., a protein or a nucleic acid. Barcodes can be as short as 2 nucleotides and as long as 100 nucleotides. A typical barcode is between 2 and 10 nucleotides long. To avoid barcode confusion due to erroneous sequencing read, barcodes used in one experiment can be designed to be an edit distance from each other as described in Levenshtein V. I., (1966) Binary codes capable of correcting deletions, insertions and reversals, Soviet Physics Doklady 10:707. In general, design and use of sample and molecular barcodes can be learned e.g., from U.S. Pat. Nos. 7,393,665, 8,168,385, 8,481,292, 8,685,678, and 8,722,368.

Single cell analysis is the current new frontier in the study of biology and disease. Recently, a new method of detecting multiple targets in individual cells termed “Quantum Barcoding” or “QBC” has been developed (U.S. Pat. No. 10,144,950). Briefly, in the QBC workflow, each cell receives a unique cell-originating combinatorial barcode that tags any antibody or probe bound to that cell. When the barcodes are decoded (e.g., by sequencing), the output contains information about the presence or co-occurrence of multiple targets (nucleic acids or proteins) in each of the millions of individual cells.

Combinatorial barcodes are assembled from subcodes via the split-pool process described in U.S. Ser. No. 10/144,950. Briefly, the process includes splitting the liquid sample into reaction volumes (e.g., wells of 96-well plate), each volume containing one species of subcode; in each reaction volume, attaching the subcode to a subcode from a previous round; and pooling the reaction volumes for a next round of attaching subcodes. More rounds of split-pool are performed until enough subcodes are attached to create the diversity of unique combinatorial barcodes sufficient to individually mark each cell in the sample. U.S. Ser. No. 10/144,950 provides a non-limiting list of specific examples of connecting the subcodes into a cell-specific code.

To date, Quantum Barcoding (QBC) has been limited to cell suspensions because the split-pool synthesis process involves mixing and distributing cells into reaction volumes. However, recently there has been increasing interest in single-cell analysis of tissue samples to obtain a two-dimensional and even three-dimensional picture of gene expression and co-expression (see e.g., WO2018091676 Method for spatial tagging and analysing nucleic acids in a biological specimen). The instant method further develops the original QBC method and adapts it to a two dimensional structure of a tissue sample.

The original QBC method has certain limitations of sensitivity, which are due to the multi-step nature of the workflow. For example, there are chemical steps of fixation and condensation as well as multiple mechanical steps applied to cells (wash, collections, filtration, centrifugations, mixing and pipetting). These steps result in cell loss limiting yield and ultimately sensitivity of the assay.

The method disclosed herein allows for single cell analysis of two-dimensional and three-dimensional samples such as tissue sections or organs. Furthermore, the method possesses robustness superior to that of the original QBC. Unlike the original QBC, the instant method enables all split-pool operations to be conducted with solid particles (such as magnetic beads) instead of cells. The advantages of using solid particles include chemical stability, ease of washing, collection and separation, to name a few. In the instant invention, the solid particles carry a location barcode that receives the same unique particle-associated combinatorial barcode as the target-binding agent (a nucleic acid probe or an antibody). As a result, the instant method detects both the presence and the location of the target within the two-dimensional or three-dimensional sample.

The present invention involves a method of handling cells in a tissue sample. In some embodiments, the sample is derived from a subject or a patient. In some embodiments the sample may comprise a fragment of a solid tissue or a solid tumor derived from the subject or the patient, e.g., by biopsy. In other embodiments, the sample is a cultured sample, e.g., a tissue culture containing cells. In some embodiments, the cells in the tissue culture spontaneously form a two-dimensional or three-dimensional structure. In some embodiments, the formation or a two-dimensional or three-dimensional structure of the tissue sample is facilitated by the addition of a matrix or an external support on which the two-dimensional or three-dimensional structure can be formed. In some embodiments, the cells of interest in the two-dimensional or three-dimensional structure are infectious agents such as bacteria, protozoa or fungi.

In some embodiments, the tissue sample is a formalin-fixed, paraffin-embedded tissue sample. Such tissue samples may be prepared by extracting tissue from a subject, exposing the tissue to a buffered formalin (or paraformaldehyde) solution, and then embedding the tissue with paraffin. The tissue sample may then be placed on a substrate, such as a microscope slide or a microarray slide. In some embodiments, the tissue sample is on a slide having a registration element suitable for identifying the sample and orientation of the sample on the slide. For example, the registration element may be a physical registration element, such as a barcode, alignment holes, alignment protrusions, alignment keys, and the like. The registration elements of the slide can facilitate orientation of the tissue sample relative to the addressable array used as described herein.

Nucleic acids, proteins or other markers of interest may be present in the cells and are the target of the cell-handling procedure. Each nucleic acid target is characterized by its nucleic acid sequence. Each protein target is characterized by its amino acid sequence and its epitopes recognized by specific antibodies. In some embodiments, the target nucleic acid contains a locus of a genetic variant, e.g., a polymorphism, including a single nucleotide polymorphism or variant (SNP of SNV), or a genetic rearrangement resulting e.g., in a gene fusion. In some embodiments, a protein biomarker contains an amino-acid change resulting in the creation of a unique epitope. In some embodiments, the target nucleic acid or target protein comprises a biomarker, i.e., a gene or protein antigen whose variants are associated with a disease or condition. For example, the target nucleic acids and proteins can be selected from panels of disease-relevant markers described in U.S. patent application Ser. No. 14/774,518 filed on Sep. 10, 2015. Such panels are available as AVENIO ctDNA Analysis kits (Roche Sequencing Solutions, Pleasanton, Cal.) In other embodiments, the target nucleic acids or proteins are characteristic of a particular organism and aids in identification of the organism or a characteristic of the pathogenic organism such as drug sensitivity or drug resistance. In yet other embodiments, the target nucleic acid or protein is a unique characteristic of a human subject, e.g., a combination of HLA or KIR sequences defining the subject's unique HLA or KIR genotype. In yet other embodiments, the target nucleic acid is a somatic sequence such as a rearranged immune sequence representing an immunoglobulin (including IgG, IgM and IgA immunoglobulin) or a T-cell receptor sequence (TCR). In yet another application, the target is a fetal sequence present in maternal blood, including a fetal sequence characteristic of a fetal disease or condition or a maternal condition related to pregnancy. For example, the target could be one or more of the autosomal or X-linked disorders described in Zhang et al. (2019) Non-invasive prenatal sequencing for multiple Mendelian monogenic disorders using circulating cell-free fetal DNA, Nature Med. 25(3):439.

In some embodiments, the target is a nucleic acid (including mRNA, microRNA, viral RNA, cellular DNA or cell-free DNA (cfDNA) including circulating tumor DNA (ctDNA)).

In some embodiments, the target is a protein expressed in the cell. For example, the protein target may be cell-surface protein. In some embodiments, the cell surface protein is a lymphocyte surface protein selected from inhibitory receptors (such as Pdcd1, Havrcr2, Lag3, CD244, Entpd1, CD38, CD101, Tigit, CTLA4), cell surface receptors (such as TNFRSF9, TNFRSF4, Klrg1, CD28, Icos, IL2Rb, IL7R) or chemokine receptors (such as CX3CR1, CCL5, CCL4, CCL3, CSF1, CXCR5, CCR7, XCL1 and CXCL10). In some embodiments, the proteins are selected from CD4, CD8, CD11, CD16, CD19, CD20, CD45, CD56 and CD279.

In some embodiments, one target is detected in the plurality of cells of the tissue sample. In other embodiments, multiple targets are detected simultaneously in the plurality of cells of the tissue sample. For example, 2, 3, 4, 5, 6, 7, 8, 9, 10, 20, 30 or more targets are detected in the plurality of cells of the tissue sample.

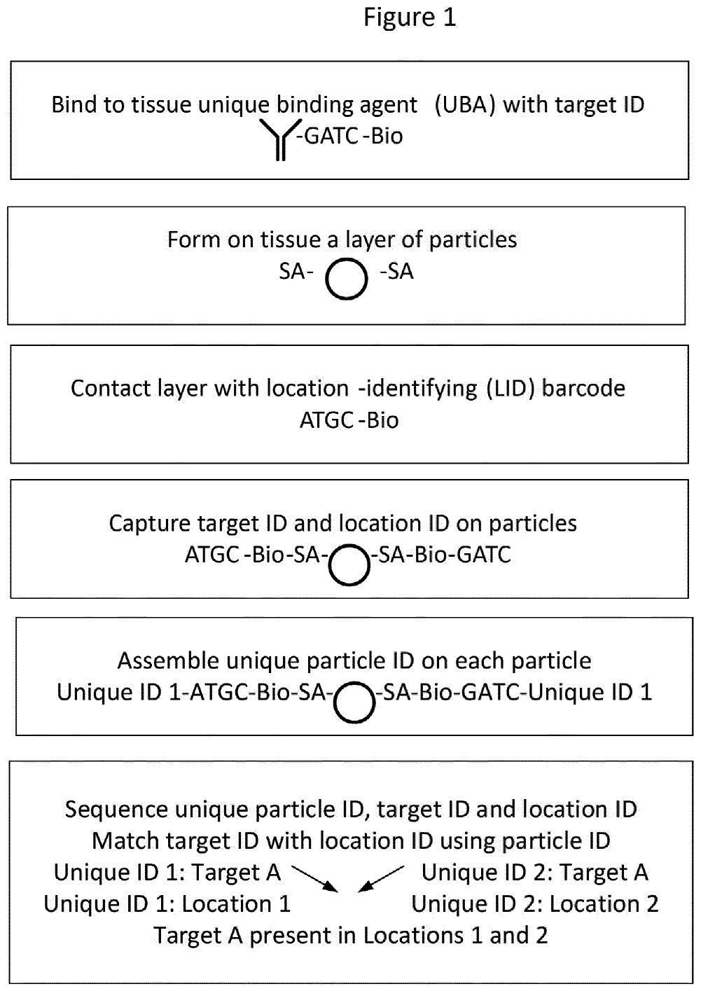

In some embodiments, the invention is a method of simultaneously detecting the presence and location of multiple targets in a tissue sample. Referring to FIG. 1, the method commences with the first step of contacting a tissue sample with one or more unique binding agents.

For ease of illustration, the unique binding agent is shown in FIG. 1 to be an antibody. However, various types of unique binding and combinations thereof may be used within the scope of the instant invention. In some embodiments, the unique binding agent is an antibody capable of specifically binding to one of the targets to be detected. The target may be a protein, a glycoprotein or a nucleoprotein present in the cell, or on the surface of the cell of the tissue sample, or in the extracellular matrix of the tissue sample. In other embodiments, the unique binding agent is a nucleic acid capable of hybridizing to a nucleic acid present in the cell of the tissue sample or in the extracellular matrix of the tissue sample. In yet other embodiments, the unique binding agent is a nucleic acid aptamer or a peptide aptamer capable of specifically binding a nucleic acid target, a protein target or a nucleoprotein target in the tissue sample.

As further illustrated in the first step of the method shown in FIG. 1, the unique binding agent comprises a target-identifying nucleic acid (shown for ease of illustration as “GATC”). In some embodiments, the unique binding agent is an antibody (immunoglobulin) and the target-identifying nucleic acid is an oligonucleotide conjugated to the antibody. Methods to attach nucleic acids to antibodies are known, e.g., Gullberg et al., PNAS 101 (22): pages 228420-8424 (2004); Boozer et al, Analytical Chemistry, 76(23): pages 6967-6972 (2004) or Kozlov et al., Biopolymers 5: 73 (5): pages 621-630 (2004).

In other embodiments, the unique binding agent is a nucleic acid (probe or aptamer) and the target-identifying nucleic acid is the target-binding sequence of the probe (or aptamer) or an additional tag sequence attached to the target-binding sequence of the probe (or aptamer).

As further illustrated in the first step of the method shown in FIG. 1, the target-identifying nucleic acid is conjugated to a capture molecule capable of selectively binding the capture moiety. For ease of illustration, the capture molecule is shown as “Bio.” In some embodiments, the capture molecule is biotin. In other embodiments, capture molecule and capture moiety are complementary oligonucleotides as described in US20200032244. In this embodiment, the capture molecule is a nucleotide sequence included in the second oligonucleotide and the fourth oligonucleotide, and the capture moiety is a complementary nucleic acid conjugated to the solid particle.

Referring to the second step illustrated in FIG. 1, the tissue sample with the unique binding agent is contacted with a layer of particles comprising a capture moiety capable of binding the capture molecule. For ease of illustration, the capture moiety is shown as “SA.” In some embodiments, the capture moiety is streptavidin and the capture molecule in biotin. In other embodiments, capture molecule and capture moiety are complementary oligonucleotides as described in US20200032244. In this embodiment, the capture molecule is a nucleotide sequence included in the second oligonucleotide and the fourth oligonucleotide described herein. The capture moiety is a complementary nucleic acid sequence conjugated to the solid particle described herein. In some embodiments, one or both of the complementary capture oligonucleotides contain one or more modified nucleotides that alter the melting temperature (Tm) of the duplex DNA. The modified nucleotides may be selected from 5-methyl cytosine, 2,6-diaminopurine, Super T (5-hydroxybutynl-2-deoxyuridine), and Super G (8-aza-7-deazaguanosine). Additionally, modified nucleotides conferring increasing the Tm include non-DNA nucleotides including locked nucleic acid (LNA) nucleotides, ribonucleotides or 2′-O-methyl ribonucleotides.

The particles form a layer on the surface of the tissue sample decorated with unique binding agents. In some embodiments, the layer is a monolayer.

Referring to the third step illustrated in FIG. 1, the tissue sample decorated with unique binding agents and layered with particles having the capture moiety is contacted with a plurality of location-identifying nucleic acids (shown for ease of illustration as “ATGC”) also conjugated to the capture molecule capable of binding the capture moiety present on the particles.

Referring to the fourth step illustrated in FIG. 1, the method comprises a step of capturing the target-identifying nucleic acids and the location-identifying nucleic acids on the particles via the capture moiety. The captured nucleic acids are now separated from the tissue sample by separating the particles from the tissue sample into a liquid sample, e.g., a fresh liquid sample.

Referring to the fifth step illustrated in FIG. 1, the method comprises a step of assembling unique particle-specific codes (“Unique ID1”) on each particle-bound target-identifying nucleic acid (shown as GATC) and each location-identifying nucleic acid (shown as ATGC) by adding to the nucleic acids a unique combination of subcode oligonucleotides. The multiple subcode oligonucleotides are added to the growing code in an ordered manner during successive rounds of split-pool synthesis performed on the particles decorated with the target-identifying nucleic acids and the location-identifying nucleic acids. Each round of the split-pool combinatorial assembly comprises splitting the liquid sample containing the particles into reaction volumes, each volume comprising a species of subcode oligonucleotide; annealing the subcode oligonucleotide adjacently to the subcode oligonucleotide from a previous round via an annealing region; in the reaction volume, covalently linking the adjacently annealed subcode oligonucleotides to each other; and pooling the reaction volumes into a liquid sample for a next round of attaching subcodes. In some embodiments, the reaction volumes are wells of a multi-well plate, e.g., an 8, 12, 96, 384 or 1536-well plate. For ease of illustration, the particle is shown as having a unique particle-specific code “Unique ID1.”

Referring further to FIG. 1, the next step is detecting the sequence of the target-identifying nucleic acids and the location-identifying nucleic acids and associated unique particle-specific codes. As shown in FIG. 1, the target-identifying and location-identifying nucleic acids acquire the same unique particle-specific code (shown as “Unique ID1”). The same unique particle-specific code allows correlating the target-identifying nucleic acids and the location-identifying nucleic acids thereby detecting both the presence and the location of each target in the tissue sample.

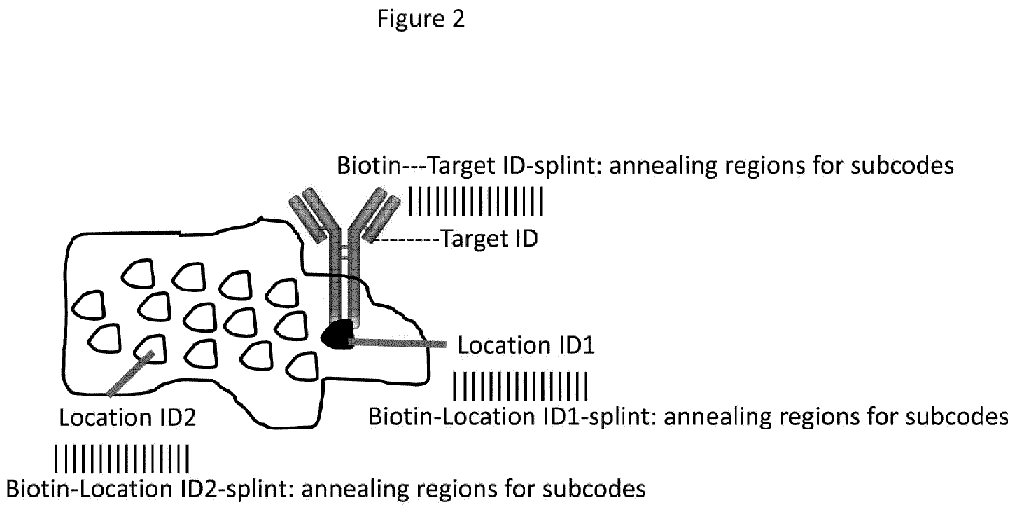

In some embodiments, the method involves a location-identifying nucleic acid and a target-identifying nucleic acid. One embodiment of the location-identifying nucleic acid and the target-identifying nucleic acid is shown in FIG. 2. Referring now to FIG. 2, the target-identifying nucleic acid comprises a first oligonucleotide including a target-identifying barcode and a second oligonucleotide hybridized to the first oligonucleotide. The embodiment illustrated In FIG. 2 involves an antibody acting as a unique binding agent with the first oligonucleotide including the target-identifying barcode conjugated thereto (e.g., by the method of Gullberg et al., (2004) PNAS 101 (22): 8420, or Boozer et al, (2004) Analytical Chemistry, 76(23):6967, or Kozlov et al., (2004) Biopolymers 5: 73 (5):621). Other embodiments involve a nucleic acid probe acting as a unique binding agent. In some embodiments where the unique binding agent is a nucleic acid probe, the first oligonucleotide is not a separate nucleic acid but a part of the probe-unique binding agent, while the second oligonucleotide hybridizes to the region in the unique binding agent including the sequence of the first oligonucleotide. In this embodiment, the target-identifying portion of the unique binding agent may be a sequence not hybridizing to the target but an additional sequence engineered into the nucleic acid probe-unique binding agent. In other embodiments where the unique binding agent is a nucleic acid probe, the first oligonucleotide including the target-identifying barcode hybridizes to the nucleic acid probe-unique binding agent, while the second oligonucleotide hybridizes to the first oligonucleotide.

Referring further to FIG. 2, the second oligonucleotide further comprises a capture molecule (shown as “Bio”). In some embodiments, the capture moiety is biotin. In other embodiments, capture molecule and capture moiety are complementary oligonucleotides as described in US20200032244. In this embodiment, the capture molecule is a nucleotide sequence included in the second oligonucleotide and the fourth oligonucleotide, and the capture moiety is a complementary nucleic acid conjugated to the solid particle.

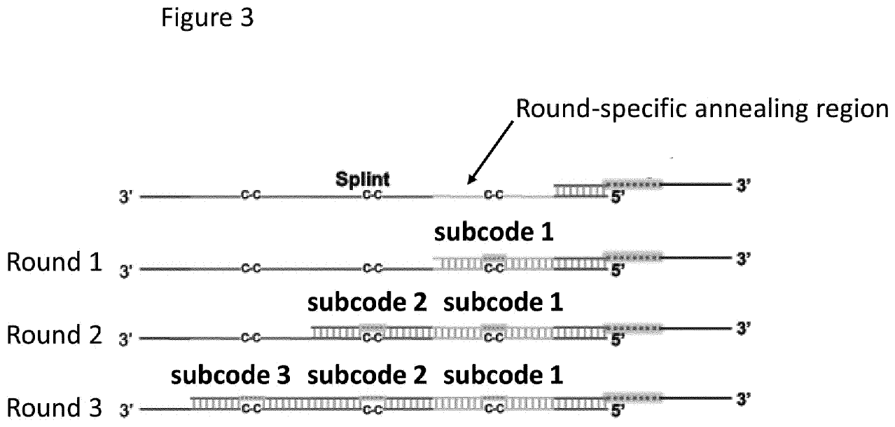

Referring further to FIG. 2, the second oligonucleotide is an anchor for assembling of the unique particle-specific codes. U.S. Pat. No. 10,144,950 (incorporated herein by reference) describes multiple strategies for assembling a unique combinatorial code (such as the particle-specific code used herein) from subcodes. As a non-limiting example, one such strategy is shown in FIG. 3.

As shown in FIG. 3, a splint oligonucleotide may be used, wherein the splint comprises annealing regions for subcode oligonucleotides. By annealing to the splint, the subcodes form the unique combinatorial barcode. For each subcode, the splint contains two annealing regions flanking a central region accommodating the diverse code regions in the plurality of subcode oligonucleotides (shown as “c-c”). In some embodiments, the central region is a non-nucleotide spacer (“carbon spacer”). In other embodiments, the central region is composed of inosine-containing nucleotides. In some embodiments of the instant invention, the second oligonucleotide includes the sequence of the splint oligonucleotide and the subcode oligonucleotides anneal to the splint portion of the second oligonucleotide. In other embodiments, the second oligonucleotide comprises an annealing region for the splint oligonucleotide and the subcode oligonucleotides anneal to the splint oligonucleotide hybridized to the second oligonucleotide (example shown in FIG. 3). Assembling codes from subcodes via a split-pool process is further described herein in a separate section.

Returning to FIG. 2, the location-identifying nucleic acid comprises a third oligonucleotide capable of attachment to the surface of the cells and including a location-identifying barcode and a fourth oligonucleotide hybridized to the third oligonucleotide.

In some embodiments, the location-identifying nucleic acid is attached directly to the cell surface. The location-identifying nucleic acid is capable of attaching to the surface of the cells when the cells are contacted with the location-identifying nucleic acid. In some embodiments, the third oligonucleotide is conjugated to a moiety capable of interacting with the surface of the cells within a tissue sample.

In some embodiments, the third oligonucleotide comprises an interacting moiety which is streptavidin. The surface of cells in the tissue sample is modified with streptavidin and the third oligonucleotide includes one or more biotinylated nucleotides. In some embodiments, streptavidin is added to the cell surface by reacting streptavidin with amino groups on the surface of the cell (e.g., epsilon amino groups of lysine in cell-membrane proteins). An effective way of conjugating streptavidin (or other avidin derivatives) to the cell surface involve maleimide activation of streptavidin and thiolation of amino groups of cell surface proteins to enable a reaction between a free sulfhydryl group and maleimide, see Espeel, P. and Du Prez, F. E. (2015) One-pot multi-step reactions based on thiolactone chemistry: a powerful synthetic tool in polymer chemistry, Eur. Polymer J. 62:247. In some embodiments, surface proteins are biotinylated and contacted with streptavidin, see Ho, V. H. B., et al, (2009) The precise control of cell labelling with streptavidin paramagnetic particles, Biomaterials 30:6548. Commercial reagents are conveniently available for streptavidin conjugation, e.g., Streptavidin Conjugation Kit—Lightning Link (Abcam, Cambridge, Mass.)

In some embodiments, the third oligonucleotide comprises an interacting moiety which is a hydrophobic moiety capable of non-covalent hydrophobic interaction i.e., insertion into a cell membrane. In some embodiments, the hydrophobic moiety is a fatty acid residue or a cholesterol moiety. For example, oligonucleotides can be joined with a palmitoyl or stearoyl residue via an amino alkyl linker. Such oligonucleotide conjugates are stably integrated into a cell membrane and form hybrids with complementary nucleic acids, see Borisenko, G., et al. (2009) DNA modification of the cell surface, Nucl. Acids Res. 37:e28.

In some embodiments, the third oligonucleotide comprises an interacting moiety which is a reactive moiety capable of forming a covalent bond with amino groups of cell membrane proteins. For example, 5′-thiol modified oligonucleotides can be conjugated to an NHS-PEG-maleimide crosslinker which reacts with available amino groups of cell membrane proteins, see Hsiao, S. C., et al., (2010) Direct Cell Surface Modification with DNA for the Capture of Primary Cells and the Investigation of Myotube Formation on Defined Patterns, Langmuir: the ACS journal of surfaces and colloids, 25(12), 6985.

In some embodiments, the third oligonucleotide comprises an interacting moiety which is a reactive moiety capable of forming a covalent bond with carbohydrates associated with cell surface proteins. For example, an oligonucleotide conjugated to a biotinylated phosphine reacts with an azido-modified sialic acid on the cell surface as described in Saxon E. and Bertozzi C. R., (2000) Cell Surface Engineering by a Modified Staudinger Reaction Science 287:2007.

Referring further to FIG. 2, the fourth oligonucleotide comprises a capture molecule (shown as “Bio”). In some embodiments, the capture moiety is biotin. In other embodiments, capture molecule and capture moiety are complementary oligonucleotides as described in US20200032244. In this embodiment, the capture molecule is a nucleotide sequence included in the second oligonucleotide and the fourth oligonucleotide, and the capture moiety is a complementary nucleic acid conjugated to the solid particle.

Referring further to FIG. 2, the fourth oligonucleotide is an anchor for assembling of the unique particle-specific codes. In reference to FIG. 3, a splint oligonucleotide may be used, wherein the splint comprises annealing regions for subcode oligonucleotides flanking a central region accommodating the diverse code regions in the plurality of subcode oligonucleotides (“c-c”). In some embodiments, the central region is a non-nucleotide spacer. In other embodiments, the central region is composed of inosine-containing nucleotides. In some embodiments, the fourth oligonucleotide includes the sequence of the splint oligonucleotide, and the subcode oligonucleotides anneal to the splint portion of the second oligonucleotide. In other embodiments, the fourth oligonucleotide comprises an annealing region for the splint oligonucleotide and the subcode oligonucleotides anneal to the splint oligonucleotide hybridized to the second oligonucleotide (example shown in FIG. 3).

Referring to FIG. 1, the invention involves the use of a solid-state particle. Many types of magnetic particles are commercially available for various laboratory applications. The particles can be magnetic polymer or silica particles coated with streptavidin or any other binding moiety. Many types of particles are commercially available. In some embodiments, the particles are streptavidin-coated silicon oxide ranging in size from 50 to 1000 nanometers (for example, the type distributed by Microspheres-Nanospheres, Cold Spring, N.Y.) In other embodiments, the particles are polymer-coated beads such as Dynabeads® (ThermoFisher Scientific, Waltham, Mass.) or magnetic glass particles (MGPs) (Roche Molecular Systems, Pleasanton, Cal.).

In some embodiments, magnetic particles are of a kind described in WO2019086517. These particles comprise a stabilizer, a superparamagnetic core, and a liquid glass coating. In some embodiments, the magnetic particle of this kind is a spherical particle 300-500 nanometers (nm) in size with the core being 270-290 nm. The particles have saturation magnetization of 50-70 Am²/kg and magnetic remanence below 3 Am²/kg. In some embodiments, the liquid glass coating is 10-20 nm thick and comprises a silicate, e.g., sodium silicate, potassium silicate, calcium silicate, lithium silicate, and magnesium silicate. In some embodiments, the magnetic core is a defined aggregate of magnetic nanoparticles<30 nm in size combined with the stabilizer such as citrate, histidine, cetyltrimethylammonium bromide (CTAB), cetyltrimethylammonium chloride (CTAC), sodium oleate or polyacrylic acid. In some embodiments, the magnetic core is Fe₃O₄, alphaFe₂O₃, gammaFe₂O₃, MnFeₓO_{y}, CoFeₓO_{y}, NiFeₓO_{y}, CuFeₓO_{y}, ZnFeₓO_{y}, CdFeₓO_{y}, BaFeₓO and SrFeₓO, wherein x is 1 to 3 and y is 3 or 4. In some embodiments, the core of the particle is Fe₃O₄.

In other embodiments, magnetic particles are of a kind described in U.S. Pat. No. 6,919,444. These magnetic particles are slightly larger spherical particles 0.5-15 micrometers (μm) in size. The particles have a magnetic core made of a magnetic metal, and a glass coating comprising one or more of SiO₂, B₂O₃, K₂O, CaO, Al₂O₃ and ZnO.

In other embodiments, magnetic particles are of a kind described in US20200041502. These magnetic particles are larger spherical superparamagnetic particles 5-40 micrometers (μm) in size and comprise a hyper-crosslinked polymer matrix covering a magnetic core. The magnetic core is comprised of 1-20 magnetic nanoparticles and have a saturation magnetization between 10 Am²/kg to 20 Am²/kg. These particles may have a pore under 100 nm in size. The polymer coating may comprise tensides, silica, silicates, silanes, phosphates, phosphonates, phosphonic acids and mixtures of two or more thereof. Alternatively, the polymer coating of such particles may comprise polyacrylic acid derivatives, tricarboxylic acids, tricarboxylic acid salts, tricarboxylic acid derivatives, amino acids, amino acid salts, amino acid derivatives, surfactants, salts of surfactants, fatty acids, fatty acid salts and fatty acid derivatives.

In some embodiments, the particles are magnetic polymer-coated particles with magnetite embedded into coating during polymerization. The polymer is formed by polymerization of divinylbenzene and vinylbenzylchloride. Some commercially available magnetic particles comprise an affinity molecule for capture of targets labeled with a ligand for the affinity molecule, e.g., streptavidin-coated particles for capture of biotinylated targets or particles adapted to the TA-PAS antibody-antigen capture system available from Biotez Berlin-Buch, GmbH, Berlin, Germany).

In other embodiments, magnetic particles are of a kind described in WO2004053490. These particles are 0.8 to 10 μm in size. The particles are magnetic polymer particles composed of a matrix polymer with pores and having superparamagnetic crystals on a surface or in the pores of the polymer and further having a polymer coating. The polymer coated is composed of two compounds selected from epoxides are selected from epichlorohydrin, epibromohydrin, isopropylglycidyl ether, butyl glycidyl ether, allylglycidyl ether, 1,4-butanediol diglycidyl ether (1,4-bis (2,3-epoxypropoxy) butane), ethylhexylglycidylether, methyl glycidylether neopentylglycol diglycidyl ether, ethylene glycol diglycidyl ether, glycerol diglycidyl ether, glycidol, and glycidyl methacrylate.

In other embodiments, magnetic particles are of a kind described in U.S. Pat. No. 9,187,691. These spherical particles are 5-8 μm in size and are composed of monodisperse epoxy coated porous matrix polymer having superparamagnetic crystals located mostly within the pores. The epoxy is selected from epichlorohydrin, epibromohydrin, isopropylglycidyl ether, butyl glycidyl ether, allylglycidyl ether, 1,4-butanediol diglycidyl ether (1,4-bis(2,3-epoxypropoxy) butane), neopentylglycol diglycidyl ether, ethylene glycol diglycidyl ether, glycerol diglycidyl ether, glycidol, glycidyl methacrylate, ethyl hexyl glycidylether, methyl glycidylether, glycerol propoxylate triglycidylether, poly(propylene glycol) didycidylether, 1,3 butanediol diglycidylether, tert butyl glycidylether, 1,4 cyclohexanedimethanol diglycidyl ether, diethylene glycol diglycidyl ether, dodecyl glycidylether, O-(2,3 epoxypropyl)-O-methylpolyethylene glycol glydidylether, glycidyl tetrafluoroethyl ether, 1,6 hexanediol diglycidylether, octyl glycidylether, decyl glycidylether, poly(epichlorohydrin-co-ethylene oxide-co-allyl glycidylether), polyethylene glycol diglycidyl ether, trimethylolethane triglycidylether, trimethylolpropane, triglycidylether, tert-butyldimethylsilyl glycidylether, 1,2-epoxybutane, 1,2-epoxypentane, 1,2-epoxy-5-hexene, 1,2-epoxy-hexane, 1,2-epoxy-7-octene, 1,2-epoxyoctane, 1,2,7,8-diepoxyoctane, 1,2-epoxy-9-decene, 1,2-epoxydecane, 1,2-epoxydodecane, and 1,2-epoxytetradecane.

In some embodiments, the particles are less than 5 micrometers in diameter, e.g., less than 1 micrometer in diameter. One of skill in the art would appreciate that the size of the particle may be chosen to accommodate the desired resolution within the tissue sample or the size of the cells in the tissue sample. In some embodiments. The layer of particles on the tissue sample is a monolayer of particles. In some embodiments, dispensing the particles is performed by a needle printer precision dispensing device such as described in U.S. Pat. No. 9,269,138. Commercially available dispensers include the cobas m511 with Bloodhound® technology (Roche Diagnostics, Indianapolis, Ind.)

In some embodiments, capture moiety on the location-identifying nucleic acid and the target-identifying nucleic acid is biotin and the particle comprises a streptavidin-coated polymer. The particle may have magnetic or paramagnetic properties. Magnetic or paramagnetic properties are especially useful for separating the particles from the tissue sample. In some embodiments, separating the particles from the tissue sample comprises denaturing the hybrids of the first and second oligonucleotides and the hybrids of the third and fourth oligonucleotides and further, separating the particles with attached nucleic acids from a solution comprising the tissue sample and any excess proteins and nucleic acids including antibodies and oligonucleotides. In some embodiments, separating the particles from the tissue sample by denaturing nucleic acid hybrids comprises the use of a formamide or formamide alternatives such as sulfolane, ethylene carbonate, pyrrolidone, DMSO or a primary amide. In some embodiments, separating the particles from the tissue sample includes a protease treatment. In some embodiments, the protease treatment is in the presence or detergents and chaotropic agents.

Referring again to the fifth step in FIG. 1, the particles undergo the split-pool process to assemble a unique particle-specific code on the nucleic acids attached to the particle. The split-pool method of assembling combinatorial barcodes is described in detail in U.S. Pat. No. 10,144,950 and O'Huallachain, M. et al. (2020) Ultra-high throughput single-cell analysis of proteins and RNAs by split-pool synthesis, Nature Commun. Biol. 3, 213, both of which are incorporated herein by reference in their entirety.

Briefly, the method comprises splitting a liquid sample comprising a plurality of particles (e.g., cells, cellular components or microparticles) into reaction volumes, e.g., wells of a 96-well plate. Each volume receives multiple particles. Each volume further comprises a species of subcode oligonucleotide. There are multiple ways of connecting together subcode oligonucleotides (see U.S. Ser. No. 10/144,950 and O'Huallachain, supra). In one embodiment illustrated in FIG. 3, the subcode oligonucleotides anneal to annealing regions in the splint oligonucleotide. The splint has multiple annealing regions, where each annealing region is round-specific to ensure that only one subcode anneals in each round. The subcode comprises round-specific annealing regions complementary to the annealing regions in the splint and further comprises the barcode. The splint accommodates diverse barcodes by containing a non-nucleotide linker or inosine-containing nucleotide to enable the formation of a stable hybrid between subcode and the splint annealed via round-specific annealing regions.

In each volume, the subcode oligonucleotide is connected to the subcode from a previous round. In some embodiments, the first subcode is connected to the second oligonucleotide or the fourth oligonucleotide in the target-identifying nucleic acid or the location-identifying nucleic acid respectively. After the subcodes have been connected to the growing code on the particles in each volume, the volumes are pooled and the particles are mixed in the pooled liquid sample. The pooled sample is again split into multiple volumes for the next round of barcode assembly.

At the completion of each round, the excess subcode oligonucleotides may optionally be removed with a double-hairpin oligonucleotide described in a U.S. Application Ser. No. 63/021,875 filed on May 8, 2020 “Removal of Excess Oligonucleotides from a Reaction Mixture.” Removal of excess subcode oligonucleotides improves yield of correctly assembled combinatorial codes and yield of post-assembly amplification reactions.

After completion of the split-pool combinatorial assembly, each particle has acquired a unique particle associated barcode. The number of rounds of split pool necessary to uniquely label each particle is the function of the number of particles and the length (and therefore diversity) or each subcode. The methods and formulas for calculating the length of a barcode and the number of cycles are disclosed in U.S. Ser. No. 10/144,950. Briefly, the number of unique particle-associated codes or tags (T) required is T=ln(1−C)/ln(1−1/N), where C is certainty of over-representation and is the number of particles. For example, with 1 million (10⁶) particles and 99.99999% (10⁻⁷) certainty of code uniqueness, one needs 16 million, or approximately 1.7×10⁷ unique particle-associated codes. To determine the number of split-pool rounds (Y) needed to achieve the desired diversity is Y=ln(T)/ln(x) where x is the number of different subcodes. If T is 1.7×10⁷ and x is 20, the number of rounds is 5.557 or approximately 6 rounds. For at least 20 different subcodes one needs to have each subcode to have a barcode of at least 3 nucleotides (43=64). Longer barcodes will have a greater editable distance apart. One of skill in the art can perform similar calculations given the input data from a particular assay.

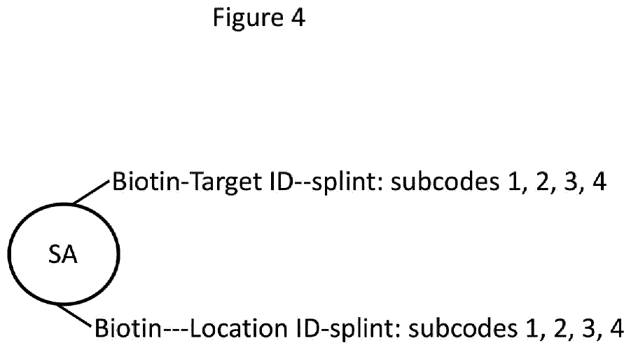

FIG. 4 illustrates a streptavidin-coated particle separated from the tissue sample, the particle having the second and the fourth oligonucleotides captured thereon. The second and the fourth oligonucleotides each have the unique particle-specific barcodes assembled thereon. The second oligonucleotide represents a target-identifying nucleic acid and the fourth oligonucleotide represents a location-identifying nucleic acid. The target-identifying nucleic acid and the location-identifying nucleic acid share the same unique particle-specific barcodes indicating that the target was present at a certain location.

In some embodiments, the invention utilizes an addressable array. In some embodiments, contacting the layer of particles with location-identifying nucleic acids comprises placing an addressable array of location-identifying nucleic acids atop the layer or particles under conditions suitable for capturing the capture moiety of the nucleic acids with capture molecule on the particles. The addressable array may be used to determine the location of the targets within the tissue sample. In some embodiments, the density of the array corresponds to the size of the cells so that each particle captures fewer than 5 cells, e.g., no more than 1 cell.

In some embodiments, multiple tissue samples are analyzed in a single experiment. In this embodiment, some steps of eh workflow are performed on each tissue sample separately. However, for gain in efficiency, some steps can be multiplexed, i.e., performed on multiple samples pooled together. In some embodiments, to enable multiplexing, the target-identifying nucleic acid further carries a sample-identifying nucleic acid barcode. Multiplexing can commence with solutions of particles with attached target-identifying nucleic acids and location-identifying nucleic acids. In some embodiments, solutions containing particles with attached target-identifying nucleic acids and location-identifying nucleic acids are pooled and together, subjected to the process of assembling the unique particle-specific barcodes. The detection step includes detecting the sequence of the target-identifying nucleic acid, the sample identifying nucleic acid and a unique particle-specific barcode, and correlating this detected sequence with the sequence of the location-identifying nucleic acid having the same unique particle-specific barcode.

In some embodiments, the particle-associated nucleic acids are separated from the particle and sequenced. These nucleic acids include the target-identifying oligonucleotides connected to a unique particle-associated combinatorial barcode, the location-identifying oligonucleotides, also connected to the same unique particle-associated combinatorial barcode and optionally, sample-identifying oligonucleotides also connected to the same unique particle-associated combinatorial barcode.

In some embodiments, the sequencing method is a high-throughput single molecule sequencing method utilizing nanopores. In some embodiments, the nucleic acids and libraries of nucleic acids formed as described herein are sequenced by a method involving threading through a biological nanopore (U.S. Ser. No. 10/337,060) or a solid-state nanopore (U.S. Ser. No. 10/288,599, US20180038001, U.S. Ser. No. 10/364,507). In other embodiments, sequencing involves threading tags through a nanopore (U.S. Pat. No. 8,461,854) or any other presently existing or future DNA sequencing technology utilizing nanopores, e.g., utilizing a device from Oxford Nanopore (Oxford, UK) selected from MinION, GridION and PromethION.

In other embodiments, sequencing is performed by other suitable technologies of high-throughput single molecule sequencing. include the Illumina HiSeq platform (Illumina, San Diego, Cal.), Ion Torrent platform (Life Technologies, Grand Island, NY), Pacific BioSciences platform utilizing the Single Molecule Real-Time (SMRT) technology (Pacific Biosciences, Menlo Park, Cal.) or any other presently existing or future DNA sequencing technology that does or does not involve sequencing by synthesis.

High throughput sequencing may utilize sequencing platform-specific primers. In some embodiments, binding sites for such primers are introduced into the nucleic acids via tailed primer extension wherein the primer comprises a sequence complementary to the nucleic acid to be sequenced and further comprises a 5′-portion (tail) containing the sequencing primer binding site. In some embodiments, the forward tailed primers are complementary to a region of the second and fourth oligonucleotide, while the reverse primers are complementary to the outer portion of the splint oligonucleotide or the final subcode oligonucleotide. In some embodiments, tailed primers are also used for pre-sequencing universal amplification.

In other embodiments, platform-specific sequencing primer binding site or universal amplification primer binding site are introduced by ligating an adaptor comprising the primer binding sites. The adaptors may be ligated to either single-stranded or double-stranded nucleic acids to be sequenced.

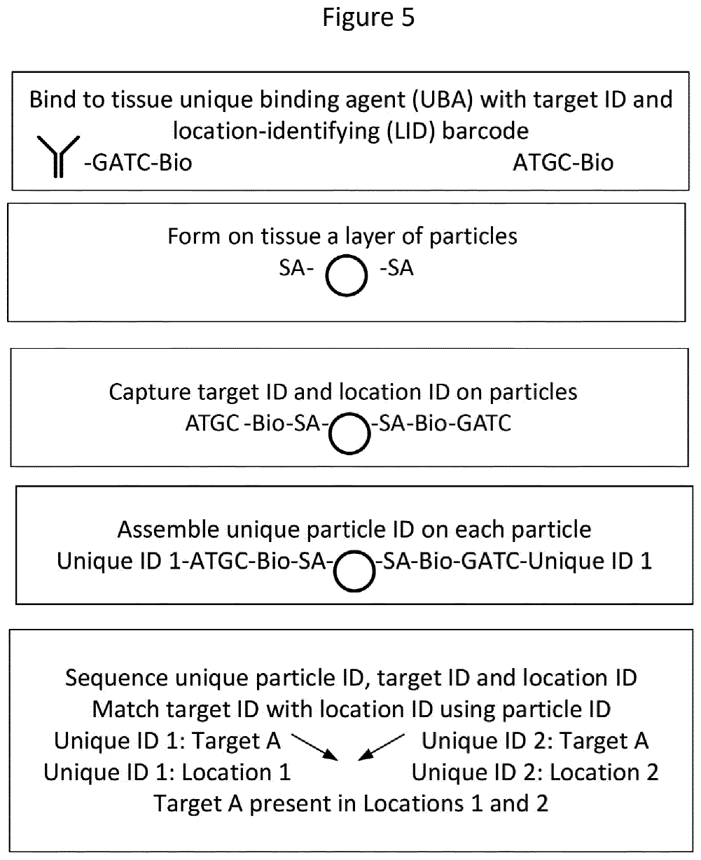

In some embodiments, the invention is an alternative method of detecting the presence and location of multiple targets in a tissue sample. The workflow of the alternative method is illustrated in FIG. 5. Referring to FIG. 5, this method comprises the first step of contacting a tissue sample with one or more unique binding agents, wherein the agents include a target-identifying nucleic acid conjugated to a capture moiety, and contacting the tissue sample with a plurality of location-identifying nucleic acids conjugated to the capture moiety. Referring to the second step in FIG. 5, the method comprises forming on the tissue sample a layer of particles conjugated to a capture molecule capable of selectively binding the capture moiety.

One of skill in the art would recognize that in this embodiment, within the first step, contacting the sample with the target-identifying nucleic acid and the location-identifying nucleic acid can occur in any sequence as long as they occur prior to the step of contacting the sample with the particles. For example, the target-identifying nucleic acid can be added first, added second or added at the exact same time as the location-identifying nucleic acid.

Referring further to FIG. 5, the next step comprises capturing the target-identifying nucleic acids and the location-identifying nucleic acids on the particles via the capture moiety. Subsequent steps are identical to the steps described in reference to FIG. 1. The steps comprise separating the particles from the tissue sample into a liquid sample. The steps further comprise assembling unique particle-specific codes on each particle-bound target-identifying nucleic acid and each particle-bound location-identifying nucleic acid. The unique particle-specific codes are assembled by adding multiple subcode oligonucleotides in an ordered manner during successive rounds of split-pool synthesis. Each round comprises: splitting the liquid sample into reaction volumes, each volume comprising a species of subcode oligonucleotide; annealing the subcode oligonucleotide adjacently to the subcode oligonucleotide from a previous round via an annealing region; covalently linking the adjacently annealed subcode oligonucleotides to each other; and pooling the reaction volumes into a liquid sample for a next round of attaching subcodes. After the subcodes are assembled, the method comprises a step of detecting the sequence of the particle-bound nucleic acids which include for each particle, the target-identifying nucleic acids associated with the unique particle-specific code and the location-identifying nucleic acids associated with the same unique particle-specific code. For each target, the method includes correlating the target-identifying nucleic acids and the location-identifying nucleic acids having the same unique particle-specific code thereby detecting the presence and location of each of the multiple targets in the tissue sample.

In the embodiment illustrated in FIG. 5, the location-identifying nucleic acid and the target-identifying nucleic acid are of the same design as shown in FIG. 2 and discussed herein in relation to the workflow example shown in FIG. 1. The target-identifying nucleic acid comprises a first oligonucleotide including a target-identifying barcode and a second oligonucleotide hybridized to the first oligonucleotide. In some embodiments, the first oligonucleotide is conjugated to an antibody-unique binding agent and includes the target-identifying barcode. In other embodiments the first oligonucleotide is a part of the nucleic acid probe-unique binding agent. In variations of this embodiment the first oligonucleotide is a part of the nucleic acid probe-unique binding agent, while the in other embodiments, the first oligonucleotide hybridizes to the nucleic acid probe-unique binding agent. The second oligonucleotide hybridizes to the first oligonucleotide and comprises a capture molecule such as biotin or a capture oligonucleotide wherein a complementary capture oligonucleotide is conjugated to the particle (see US20200032244).

The second oligonucleotide is also an anchor for assembling of the unique particle-specific codes. In some embodiments, the code is assembled via a splint oligonucleotide as shown in FIG. 3 and discussed herein in relation to the workflow example shown in FIG. 1. The splint comprises annealing regions for subcode oligonucleotides, comprising for each subcode, two annealing regions flanking a central region accommodating the diverse code regions in the plurality of subcode oligonucleotides. The central region may be a non-nucleotide spacer or be composed of inosine-containing nucleotides. In some embodiments, the second oligonucleotide includes the sequence of the splint oligonucleotide, and the subcode oligonucleotides anneal to the splint portion of the second oligonucleotide. In other embodiments, the second oligonucleotide comprises an annealing region for the splint oligonucleotide and the subcode oligonucleotides anneal to the splint oligonucleotide hybridized to the second oligonucleotide.

The location-identifying nucleic acid is also illustrated in FIG. 2 and comprises a third oligonucleotide capable of attachment to the surface of the cells and including a location-identifying barcode and a fourth oligonucleotide hybridized to the third oligonucleotide.

In some embodiments, the location-identifying nucleic acid is attached directly to the cell surface by means of the third oligonucleotide comprising an interacting moiety interacting with the surface of cells in the tissue sample. The interacting moiety may be biotin where cells are pre-treated with streptavidin. The interacting moiety may be a hydrophobic moiety (e.g., palmitoyl or stearoyl residue or a cholesteryl moiety conjugated to the oligonucleotide via an amino alkyl linker) capable of non-covalent hydrophobic interaction i.e., insertion into a cell membrane. The interacting moiety may be a reactive moiety capable of forming a covalent bond with amino groups of cell membrane proteins, e.g., an NHS-PEG-maleimide crosslinker which reacts with available amino groups of cell membrane proteins. The interacting moiety may also be a reactive moiety capable of forming a covalent bond with carbohydrates associated with cell surface proteins, e.g., a biotinylated phosphine reacting with an azido-modified sialic acid on the cell surface.

The fourth oligonucleotide hybridized to the third oligonucleotide comprises a capture molecule, e.g., biotin or a capture oligonucleotide wherein a complementary capture oligonucleotide is conjugated to the particle (see US20200032244). The fourth oligonucleotide is also an anchor for assembling of the unique particle-specific codes acting in the same way as the second oligonucleotide described above.

The method shown in FIG. 5 utilizes the same type of a solid-state particle as described in relation to the method in FIG. 1. The particle may be less than 5 micrometers in diameter or less than 1 micrometer in diameter and may be selected to accommodate the desired resolution within the tissue sample or the size of the cells in the tissue sample. In some embodiments. The layer of particles on the tissue sample is a monolayer of particles. In some embodiments, dispensing the particles is performed by a needle printer precision dispensing device, e.g., cobas m511 with Bloodhound® technology (Roche Diagnostics, Indianapolis, Ind.)

In some embodiments, the capture moiety on the location-identifying nucleic acid and the target-identifying nucleic acid is biotin and the particle comprises a streptavidin-coated polymer coating a metal with magnetic or paramagnetic properties useful for separating the particles from the tissue sample. In some embodiments, separating the particles from the tissue sample comprises denaturing the hybrids of the first and second oligonucleotides and the hybrids of the third and fourth oligonucleotides and further, separating the particles with attached nucleic acids from a solution comprising the tissue sample and any excess proteins and nucleic acids including antibodies and oligonucleotides. In some embodiments, the method further comprises washing the solution comprising separated particles and placing the particles into a new solution for assembling unique particle-specific codes. In some embodiments, separating the particles from the tissue sample by denaturing nucleic acid hybrids comprises the use of a formamide or formamide alternatives such as sulfolane, ethylene carbonate, pyrrolidone, DMSO or a primary amide. In some embodiments, separating the particles from the tissue sample includes a protease treatment. In some embodiments, the protease treatment is in the presence or detergents and chaotropic agents.

As in the method in FIG. 1, the particles undergo the split-pool process to assemble a unique particle-specific code on the nucleic acids attached to the particle to form a structure shown in FIG. 4.

In one embodiment, the invention is a kit including reagents for performing the novel method of simultaneously detecting the presence and location of multiple targets in a tissue sample disclosed herein. The kit comprises one or more unique binding agents, wherein the agents include a target-identifying nucleic acid conjugated to a capture moiety; solid state particles conjugated to a capture molecule capable of selectively binding the capture moiety; a plurality of location-identifying nucleic acids conjugated to the capture moiety; and a plurality of subcode oligonucleotides comprising annealing region and a code and reagents for connecting the subcode to each other. In some embodiments, the unique binding agent in the kit is an antibody conjugated to the target-identifying nucleic acid. In other embodiments, the unique binding agent in the kit is a nucleic acid probe comprising a target-identifying barcode. In some embodiments, the target-identifying nucleic acid in the kit comprises a first oligonucleotide including a target-identifying barcode; and a second oligonucleotide hybridized to the first oligonucleotide and including the capture moiety and further including the annealing region for attaching subcodes. Similarly, the location-identifying nucleic acid in the kit comprises a third oligonucleotide capable of attachment to the surface of the cells and including a location-identifying barcode; and a fourth oligonucleotide hybridized to the third oligonucleotide and including the capture moiety and further including the annealing region for attaching subcodes.

If multiple samples are to be pooled, the kit further comprises a sample-identifying oligonucleotide conjugated to a moiety capable of attaching to the surface of the cells in the tissue sample. The moiety is selected from biotin, a fatty acid residue or a cholesterol moiety capable of forming a hydrophobic interaction with the membrane of the cells, a maleimide moiety capable of reacting with amino groups present in cell membrane proteins of the cells or a phosphine moiety capable of reacting with carbohydrate residues associated with cell membrane proteins of the cells.

In some embodiments, the particles in the kit comprise a streptavidin-coated polymer with a core or additive conferring magnetic or paramagnetic properties. The particles in the kit are 1, 2, 3, 4, 5, 6, 7, 8, 9, or about 10 micrometers in diameter.

The kit may further comprise washing reagents. For example, for removing proteins and other tissue remnants, the kit may include a protease, a detergent or a chaotropic agent. For denaturing nucleic acids, the kit may comprise formamide or formamide alternatives such as sulfolane, ethylene carbonate, pyrrolidone, DMSO or a primary amide.

In some embodiments, the location-identifying oligonucleotides in the kit are supplied in an addressable array.

If multiple samples are to be pooled, the kit further comprises sample-identifying nucleic acids. These can be present as oligonucleotides, biotinylated oligonucleotides, oligonucleotides with modifications permitting conjugation to the surface of cells or oligonucleotides incorporated into location-identifying or target-identifying oligonucleotides.

In some embodiments, the invention is a system for detecting the presence and location of multiple targets in a tissue sample. The system comprises a computer for executing one or more computer programs. The computer programs comprise computer code enabling collecting and analyzing data on barcodes associated with an addressable array, including location and identity of barcodes associated with each location in the array. The computer programs further comprise computer code enabling collecting and analyzing data on barcodes associated with unique binding agents, including identity of barcodes associated with each unique binding agent. The computer programs further comprise computer code enabling collecting and analyzing data on barcodes associated with each particle, including identity of barcodes associated with each particle and each nucleic acid associated with the particle. The computer programs further comprise computer code enabling collecting and analyzing data to establish a correlation between each unique particle associated barcode and target-identifying nucleic acid and location identifying nucleic acid associated with the particle. The computer programs further comprise computer code enabling a graphic display of information indicating the location of each target in the tissue sample.

Specifically, the computer programs comprise computer code enabling associating each location-identifying nucleic acid with a location on the array, and further associating each target-identifying nucleic acid with the location-identifying nucleic acid by virtue of sharing a unique particle-associated barcode, thus associating the target with the location in the array. The computer programs further comprise computer code enabling superimposing of the addressable array onto the tissue sample, generating an image comprising the tissue sample, the array and the location of each target in the array.

The computer includes one or more memory storage devices and a programmable processor. To memory storage elements may include one or more storage elements, such as volatile memory, non-volatile memory, read-only memory (ROM), random access memory (RAM), or the like. The memory storage device can be one or more physical apparatuses used to store data or programs on a temporary or permanent basis. In some instances, the device is volatile memory and requires power to maintain stored information. In other instances, the device is non-volatile memory and retains stored information on a storage device including, by way of non-limiting examples, CD-ROMs, DVDs, flash memory devices, magnetic disk drives, magnetic tapes drives, optical disk drives, and cloud computing-based storage. The system further comprises a graphic display to display the images generated by the computer, including the image comprising the tissue sample, the array and the location of each target in the array.

### Examples

### Example 1 (Prophetic). Simultaneously Determining the Presence and Location of Targets in a Two-Dimensional Tissue Sample

In this example, a fresh-frozen tissue is sectioned for staining with antibodies. The fresh-frozen tissue sample is equilibrated to −20° C. and sliced into 5 μm sections. The tissue slices are fixed by immersion in cold acetone (−20° C.) for 2 minutes and allowed to air dry at room temperature. The slides are rinsed three times in PBS, to remove the tissue-freezing matrix. Optionally, the slides are treated to block non-specific binding by incubating with blocking buffer comprising a dilution of serum from antibody host species.

A mixture of antibodies is provided where antibodies are pre-conjugated to first oligonucleotides including antibody-identifying barcodes (10-20 nucleotides long) and a second oligonucleotide is hybridized to the first oligonucleotide. The second oligonucleotide contains a complement of the antibody-identifying barcode and a splint for assembling a combinatorial barcode. The splint includes annealing regions for four rounds of subcode annealing. Annealing regions are how long and are separated by a non-nucleotide linker that accommodates a how long? Barcode in each subcode oligonucleotide. The second oligonucleotide also contains one or more biotinylated nucleotides.

The antibody is applied to the tissue sections on the slide under standard immunostaining conditions for 1 hour at room temperature followed by a wash with standard wash buffers containing non-denaturing non-ionic detergents and sodium citrate salts (e.g., wash buffer 1 (0.4×SSC/0.3% IGEPAL®, pH 7) and wash buffer 2 (2×SSC/0.1% IGEPAL®, pH 7).

The tissue sample stained with antibodies is contacted with streptavidin-coated magnetic beads Dynabeads™ M-280 Streptavidin (ThermoFisher Scientific, Waltham, Mass.) The beads are spread to form a monolayer using a needle printer system (e.g., cobas m 511 analyzer using Bloodhound® technology, Roche Diagnostics, Indianapolis, Ind.). Biotinylated oligonucleotides comprising the target-identifying barcode become associated with the streptavidin-coated beads.

Next, the monolayer of beads is contacted with a third oligonucleotide including location-identifying barcode (10-20 nucleotides long) and a fourth oligonucleotide hybridized to the third oligonucleotide. The fourth oligonucleotide contains a complement of the location-identifying barcode and a splint for assembling a combinatorial barcode identical to the splint on the second oligonucleotide. The fourth oligonucleotide also contains one or more biotinylated nucleotides. Biotinylated oligonucleotides comprising the location-identifying barcode also become associated with the streptavidin-coated layer of beads. The printing process is accomplished by DNA-directed immobilization on high-density DNA arrays whereas the original high-density DNA array may be used to pick up and spatially order the library of sequences. The transfer of biotinylated oligonucleotides is accomplished by hybridization-denaturation. The quick association of biotinylated oligonucleotides with the streptavidin-coated beads limits any misalignment to a minimum so that each bead is associated with 6-12 location-identifying barcodes. The transfer of probes to beads is facilitated by heating and gently pushing up-down the source of the magnetic field.

Next, the hybrids between the first and second oligonucleotides and the hybrids between the third and fourth oligonucleotides are denatured. Only the second (target-identifying) and the fourth (location-identifying) oligonucleotides remain associated with the beads via biotin-streptavidin interaction. The beads are collected and washed thoroughly. All proteins are quantitatively removed from the beads and the solution including the beads by enzymatic (protease) digestion in the presence of chaotropic reagents or detergents.

Optionally, multiple samples may now be combined. Biotinylated sample identifying barcodes can be added to beads in each sample at any step prior to this step. Alternatively, sample-identifying barcodes may be added to location identifying oligonucleotides (e.g., the fourth oligonucleotide).

Next, the beads associated with the target-identifying oligonucleotides, the location-identifying oligonucleotides and optionally, the sample-identifying oligonucleotides are subjected to a split-pool protocol of assembling combinatorial barcodes essentially as described in O'Huallachain, M. et al. (2020) Ultra-high throughput single-cell analysis of proteins and RNAs by split-pool synthesis, Nature Commun. Biol. 3, 213. Each bead is now associated with the following types of nucleic acids: the target-identifying oligonucleotides connected to a unique particle-associated combinatorial barcode, the location-identifying oligonucleotides, also connected to the same unique particle-associated combinatorial barcode and optionally, sample-identifying oligonucleotides also connected to the same unique particle-associated combinatorial barcode.

Next, the nucleic acids are separated from beads and sequenced with an optional pre-sequencing amplification step. Amplification and sequencing are accomplished via designing universal primer binding sites and sequencing primer binding sites into the target-identifying oligonucleotides and the location-identifying oligonucleotides (and optionally, sample-identifying oligonucleotides) on one side, and the splint or the last subcode oligonucleotides on the other side. Universal amplification is conducted with any commercial PCR reagents and instruments (e.g., from KAPA Biosystems, Roche Sequencing and Life Science, Wilmington, Mass.). The amplified nucleic acids are separated from the PCR reagents e.g., via SPRI bead purification (Beckman Coulter, Irvine, Cal.) Purified amplification products are sequenced, e.g., on a MiSeq instrument or another sequencing instrument from Illumina or any alternative sequencing platform.

The sequence of the nucleic acid allows correlating the target-identifying oligonucleotides and the location-identifying oligonucleotides (and optionally, the sample-identifying oligonucleotides) that are associated with the same unique particle-associated combinatorial barcode. The correlation signifies the presence of the target at a location on the addressable array used to distribute location-identifying nucleic acids.

1. A kit for simultaneously detecting the presence and location of multiple targets in a tissue sample, the kit comprising:
a. one or more unique binding agents, wherein the agents include a target-identifying nucleic acid conjugated to a capture moiety;
b. solid state particles conjugated to a capture molecule capable of selectively binding the capture moiety;
c. a plurality of location-identifying nucleic acids conjugated to the capture moiety;
d. a plurality of subcode oligonucleotides comprising annealing region and a code and reagents for connecting the subcode to each other.

2. The kit of claim 1, wherein the unique binding agent is an antibody conjugated to the target-identifying nucleic acid.

3. The kit of claim 1, wherein the unique binding agent is a nucleic acid probe comprising a target-identifying barcode.

4. The kit of claim 1-3, wherein the target-identifying nucleic acid comprises:
a. a first oligonucleotide including a target-identifying barcode; and
b. a second oligonucleotide hybridized to the first oligonucleotide and including the capture moiety and further including the annealing region for attaching subcodes.

5. The kit of claim 1-4, wherein the location-identifying nucleic acid comprises:
a. a third oligonucleotide capable of attachment to the surface of the cells and including a location-identifying barcode; and
b. a fourth oligonucleotide hybridized to the third oligonucleotide and including the capture moiety and further including the annealing region for attaching subcodes.

6. The kit of claim 1-5, further comprising a sample-identifying oligonucleotide conjugated to a moiety capable of attaching to the surface of the cells in the tissue sample.

7. The kit of claim 1-6, wherein the capture moiety is biotin and the particle comprises a streptavidin-coated polymer.

8. The kit of claim 1-7, wherein the particle has magnetic or paramagnetic properties.

9. The kit of claim 1-9, wherein the particles are spheres fewer than 10 micrometers in diameter.

10. The kit of claim 1-9, further comprising a protease, a detergent and a chaotropic agent.

11. The kit of claim 1-10, wherein the location-identifying oligonucleotides are supplied in an addressable array

12. The kit of claim 1-11, further comprising a solution of formamide or formamide alternatives selected from sulfolane, ethylene carbonate, pyrrolidone, DMSO or a primary amide.

13. The kit of claim 1-12, wherein the unique binding agent further comprises a sample-identifying nucleic acid and multiple liquid samples are pooled prior to detecting particle-specific barcodes.

14. The kit of claim 1-13, further comprising a double-hairpin oligonucleotide for removing excess subcodes from the reaction mixture, the double hairpin nucleic acid comprising a single nucleic acid strand having:
i. a first hairpin at the 5′-end;
ii. a second hairpin at the 3′-end; and
iii. a single-stranded region between the 5′-end and the 3′-end, wherein the single-stranded region comprises a sequence capable of hybridizing to the subcode oligonucleotide.

15. The kit of claim 1-14, wherein the capture moiety is a capture sequence on the second and fourth oligonucleotides and the kit comprises particles conjugate to a capture molecule comprising a capture oligonucleotide complementary to the capture sequence.

16. A method of simultaneously detecting the presence and location of multiple targets in a tissue sample, the method comprising:
a. contacting a tissue sample with one or more unique binding agents, wherein the agents include a target-identifying nucleic acid conjugated to a capture moiety;
b. forming on the tissue sample a layer of particles conjugated to a capture molecule capable of selectively binding the capture moiety;
c. contacting the layer of particles with a plurality of location-identifying nucleic acids conjugated to the capture moiety;
d. capturing the target-identifying nucleic acids and the location-identifying nucleic acids on the particles via the capture moiety and separating the particles from the tissue sample into a liquid sample;
e. assembling unique particle-specific codes on each particle-bound target-identifying nucleic acid and each location-identifying nucleic acid by adding to the nucleic acids multiple subcode oligonucleotides in an ordered manner during successive rounds of split-pool synthesis wherein each round comprises:
i. splitting the liquid sample into reaction volumes, each volume comprising a species of subcode oligonucleotide;
ii. annealing the subcode oligonucleotide adjacently to the subcode oligonucleotide from a previous round via an annealing region;
iii. covalently linking the adjacently annealed subcode oligonucleotides to each other; and
iv. pooling the reaction volumes into a liquid sample;
f. detecting the sequence of the target-identifying nucleic acids and the location-identifying nucleic acids and associated unique particle-specific codes;
g. for each target, correlating the target-identifying nucleic acids and the location-identifying nucleic acids having the same unique particle-specific code thereby detecting the presence and location of multiple targets in the tissue sample.

17. A method of detecting the presence and location of multiple targets in a tissue sample, the method comprising:
a. contacting a tissue sample with
i. one or more unique binding agents, wherein the agents include a target-identifying nucleic acid conjugated to a capture moiety;
ii. a plurality of location-identifying nucleic acids conjugated to the capture moiety;
b. forming on the tissue sample a layer of particles conjugated to a capture molecule capable of selectively binding the capture moiety;
c. capturing the target-identifying nucleic acids and the location-identifying nucleic acids on the particles via the capture moiety and separating the particles from the tissue sample into a liquid sample;
d. assembling unique particle-specific codes on each particle-bound target-identifying nucleic acid and each location-identifying nucleic acid by adding to the nucleic acids multiple subcode oligonucleotides in an ordered manner during successive rounds of split-pool synthesis wherein each round comprises:
i. splitting the liquid sample into reaction volumes, each volume comprising a species of subcode oligonucleotide;
ii. annealing the subcode oligonucleotide adjacently to the subcode oligonucleotide from a previous round via an annealing region;
iii. covalently linking the adjacently annealed subcode oligonucleotides to each other; and
iv. pooling the reaction volumes into a liquid sample;
e. detecting the sequence of the target-identifying nucleic acids and the location-identifying nucleic acids and associated unique particle-specific codes;
f. for each target, correlating the target-identifying nucleic acids and the location-identifying nucleic acids having the same unique particle-specific code thereby detecting the presence and location of multiple targets in the tissue sample.

18. The method of claim 16-17, further comprising a step of removing excess subcode oligonucleotides from a reaction mixture, the method comprising:
a. after attaching the subcode oligonucleotides to the subcode oligonucleotide of the previous round, contacting the reaction mixture with a double hairpin nucleic acid comprising a single nucleic acid strand having:
i. a first hairpin at the 5′-end;
ii. a second hairpin at the 3′-end; and
iii. a single-stranded region between the 5′-end and the 3′-end, wherein the single-stranded region comprises a sequence capable of hybridizing to the subcode oligonucleotide;
b. annealing the excess subcode oligonucleotide to the double hairpin nucleic acid;
c. ligating the excess subcode oligonucleotide to the ends of the double hairpin nucleic acid thereby removing the excess subcode oligonucleotide from the reaction mixture.
